(19) United States
(12) Patent Application Publication
(10) Pub. No.: US 2003/0108630 A1
(43) Pub. Date: Jun. 12, 2003

(54) Morinda citrifolia enhanced naturaceutical formulation and method for treating and preventing migraine headaches

(76) Inventors: Stephen Story, Alpine, UT (US); Claude Jarakae Jensen, Cedar Hills, UT (US); Afa Kehaati Palu, Orem, UT (US)

Correspondence Address: KIRTON & McCONKIE, 1800 Eagle Gate Tower, 60 East South Temple, Salt Lake City, UT, 84111, US

(21) Appl. No.: 10/285,287

(22) Filed: Oct. 31, 2002

Related U.S. Application Data
(60) Provisional application No. 60/335,321, filed on Nov. 2, 2001.

(51) Int. Cl. A61K 35/78

(52) U.S. Cl. 424/765; 424/777

(57) ABSTRACT

The present invention features methods and formulations or compositions, such as a naturaceutical formulation, for treating and preventing headaches, and particularly migraine headaches, as well as methods and formulations for treating the conditions and symptoms often associated with migraine headaches. The naturaceutical formulations comprise an identified amount of a processed Morinda citrifolia product by weight, and the method comprises the prophylactic administration of the processed Morinda citrifolia product-based naturaceutical formulation in a safe, pre-determined or identified amount for a safe, pre-determined frequency, for a safe, pre-determined duration of time.

### RELATED APPLICATIONS

This application claims priority to U.S. Provisional Application Serial No. 60/335,321, filed Nov. 2, 2001, entitled, “Methods for Treating Symptoms Associated with Migraine Headaches.”

### BACKGROUND

1. Field of the Invention

The present invention relates to methods and naturaceutical formulations and substances for treating and preventing headaches, and particularly migraine headaches, and their associated symptoms and conditions. Specifically, the present invention relates to Morinda citrifolia-based methods and formulations for treating and relieving pre-existing headaches, as well as to methods and formulations for preventing the onset or reducing the onset potential of additional, future headaches. The present invention is particularly suited for treatment and prevention of migraine headaches as commonly experienced in mammals, and particularly humans.

2. Background of the Invention and Related Art

The lifetime prevalence of migraine headaches is consistent around the world ranging from 6% to 12% in men and 15% to 25% in women. The maximum prevalence occurs between the ages of 30 and 50 in both men and women and can persist through the age of 70 to 80. Very often migraine headaches are responsive to standard medical intervention, but not always. As many as 73% of patients have frequent low-grade headaches between attacks and these are increasingly recognized as having migraine origin as well. The costs in terms of human suffering and economic losses are large: an average of $817 is spent in the United States per year on direct treatment. Approximately 270 working days per year are lost through inability to work for every 1000 migraine sufferers in the work force. In addition, migraine headaches are now recognized to contribute to a number of comorbid disorders over life including panic disorder, anxiety disorder and affective disorders in the younger, and hypertension, myocardial infarction and stroke in the older patient populations. While medications can be effective, their overuse is also attributed to be the cause of drug-induced refractory headaches. Some patients seeking relief will turn to alternative therapies such as chiropractic, acupuncture and naturaceuticals (herbs, vitamin and other natural food supplements).

As with every disease, the earlier they can be identified, the earlier their cause can be determined. Early detection results in treatment. Unlike other diseases however, migraine headaches do not seem to follow this general deduction as there exists several factors or conditions that can trigger a migraine headache or the symptoms of a migraine headache.

Migraine headaches originate in the cerebrum of the brain. Some experts believe that a migraine headache is a result of a series of events that begins in one area of the brain as an electrical charge, which in turn causes alterations in another area of the brain especially in the trigeminovascular system. The changes that occur in the electrical signals of the brain lead to biochemical changes in the brain, the resultant effect of which is clumping of the platelets, alterations in the size of blood vessels, and release of pain-producing substances, such as serotonin or other related substances.

The term migraine is defined herein as a severe recurring headache resulting from cerebral vasoconstriction contributing to subsequent cerebral vasodilatation and is classified as either a classical migraine or a common migraine. See, e.g., “Remington's Pharmaceutical Sciences”, 17th ed., Mack Publishing Company (1985), p. 946 and Goodman and Gilman's “The Pharmaceutical Basis Of Therapeutics”, 8th ed., McGraw-Hill, Inc.(1990), pp. 944-947. A common migraine is much more likely to occur than a classical migraine.

The classical migraine is associated with objective prodromal neurological signs and symptoms involving a headache that is preceded by a slowly expanding area of blindness surrounded by a sparkling edge that increases to involve up to one half of the field of vision of each eye. When the blindness clears up after approximately 20 minutes, it is often followed by a severe one-sided headache with nausea, vomiting and sensitivity to light.

The common migraine is an attack without prodromal symptoms and begins as a slowly developing pain in the form of a headache that transforms into a mounting throbbing pain made worse by the slightest movement or noise. The pain is often on one side of the head only and usually occurs with nausea and sometimes vomiting.

The typical duration of time that migraine lasts ranges from about two hours to two days. Examples of causes of migraine headaches are: stress related (e.g., anxiety, anger, worry, excitement, shock, depression, overexertion, changes of routine and changes of climate), food-related (e.g., chocolate, cheese and other dairy products, red wine, fried food and citrus fruits), sensory-related (e.g., bright lights or glare, loud noises and intense or penetrating smells, menstruation and contraceptive drugs). However, making treatment more difficult, not all migraines originate due to a particular internal or external environmental condition. Some migraine sufferers claim that their migraine headaches were triggered as a result of particular foods eaten, especially those that contain caffeine, while others claim that their migraine headache is a result of depression or severe anxiety.

As such, treatments for migraine and other headaches are numerous, including both narcotic or medicinal treatments, as well as various forms of homeopathic and other similar treatments. However, medication is not the only key to relieve migraine headaches. An improved lifestyle full of balanced nutrition and regular exercise will also help to prevent the onset of migraine headaches.

Medications are typically categorized into preventative medications or pain killing medications. Pain medications function to interfere with the process that causes the headache in order to stop the pain. Some medications are effective but their side effects shadows their efficacy while others will lead the sufferer to total dependency. An example of some anti-migraine drugs include: U.S. Pat. Nos. 4,650,810, 4,914,125, 4,916,125, 4,994,483, 5,021,428,5,200,413, 5,242,949, 5,248,684, 5,273,759, 5,317,103, 5,364,863, 5,399,574, 5,434,154, 5,441,969, 5,464,864, 5,466,699, 5,468,768, 5,491,148 and 5,494,910.

Anti-migraine drugs most commonly used in treatment of migraine headaches typically fall into the following groups: ergot alkaloids, beta-blocking agents, calcium channel blocking agents, antidepressants, selective 5-HT.sub.1 agonists (sumatriptan), sedatives, local anesthetics, adrenergic blocking agents, and mixtures of these.

Management of migraine headaches is complicated for several reasons, including the lack of a single therapy which is effective in all patients with the same type of migraine and by the need to select either an abortive or prophylactic method of treatment. Further complications involve use of some drugs that can cause dependence with extended use, such as the ergot alkaloid Ergotamine. Another important consideration is that many of the more effective anti-migraine agents that are currently in use (e.g., the ergots, methysergide, etc.) tend to produce severe side effects with long term or continued usage.

### SUMMARY OF THE INVENTION

In response to the deficiencies in prior art formulations and remedies for treatment and prevention of migraine headaches, the present invention features unique natural methods and formulations for treating migraine headaches and headaches in general.

Therefore, it is an object of some embodiments of the present invention to provide a naturaceutical formulation for treating migraine headaches and headaches in general, as well as the symptoms and conditions associated with migraine headaches.

It is another object of some embodiments of the present invention to provide a naturaceutical formulation or composition comprising one or more processed Morinda citrifolia products.

It is still another object of some embodiments of the present invention to provide one or more methods of administering the naturaceutical formulation to a mammal to treat an existing headache or inhibit or prevent the onset of additional future headaches.

In accordance with the invention as embodied and broadly described herein, the present invention features a processed Morinda citrifolia product embodied in one or more forms of a naturaceutical composition or formula to reduce the severity, frequency and duration of migraine headaches, to treat the symptoms of headaches, and to reduce or prevent the onset of future headaches.

In some exemplary embodiments, the processed Morinda citrifolia product may comprise processed Morinda citrifolia fruit juice, processed Morinda citrifolia puree juice, processed Morinda citrifolia dietary fiber, processed Morinda citrifolia oil or oil extract, or processed Morinda citrifolia fruit juice or puree juice in concentrate form. The naturaceutical formulation may be embodied in one of several forms, such as a pill, a lozenge, a beverage, a powder dring mix, a gel capsule, a syrup, or any other known form providing the ability of delivering the processed processed Morinda citrifolia product or active ingredient of a processed Morinda citrifolia product to the body of a mammal.

The present invention further teaches the method of administring the naturaceutical formulation or composition comprising the one or more processed Morinda citrifolia products, preferably processed Morinda citrifolia fruit juice or puree or puree juice, to both treat established or pre-existing headaches and the conditions or symptoms associated therewith, as well as to reduce the onset potential of additional, future headaches. The Morinda citrifolia fruit juice or puree or puree juice comprises an affective amount of several active ingredients to provide a non-toxic carrier medium.

In still another embodiment, the present invention features a method of administering a naturaceutical formulation comprising one or more processed Morinda citrifolia products concurrently with a migraine medication. Taking the Morinda citrifolia-based naturaceutical concurrently with a migraine medication functions to increase the efficacy of the migraine medication.

### DETAILED DESCRIPTION OF THE PREFERRED EMBODIMENTS

It will be readily understood that the components of the present invention, as generally described herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The presently preferred embodiments of the invention will be best understood by separating the description into sections, the first pertaining to a general discussion regarding Morinda citrifolia, including its origins, processing techniques, and health benefits, and the methods employed to produce and manufacture the processed Morinda citrifolia products used as key ingredients in the naturaceutical formulations described herein; and the second being a more detailed and specific discussion on the Morinda citrifolia-based methods and naturaceutical formulations or compositions used to treat and prevent migraine headaches and their associated symptoms or conditions, such treatment methods involving the prophylactic administration of the processed Morinda citrifolia products as described herein. Examples of experimental studies and the results obtained are also provided herein.

### General Discussion of Morinda Citrifolia and the Methods Used to Produce Processed Morinda Citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in food products used to treat migraine headaches and its related symptoms. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention utilizes the fruit juice, the puree, and the oil extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product in concentrate or dilute (such as with water or other fruit juices) form. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp a separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment may include a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp may be pasteurized at a temperature of 181° F. (83° C.) minimum and then packed in drums for further processing or made into a high fiber product.

Drying may further process the wet pulp. The methods of drying may include freeze-drying, drum drying, tray drying, sun drying, and spray drying. The dried Morinda citrifolia pulp may include a moisture content in the range from 0.1 to 15 percent by weight and more preferably from 5 to 10 percent by weight. The dried pulp preferably has a fiber content in the range from 0.1 to 30 percent by weight, and more preferably from 5 to 15 percent by weight.

The high fiber product may include wet or dry Morinda citrifolia pulp, supplemental fiber ingredients, water, sweeteners, flavoring agents, coloring agents, and/or nutritional ingredients. The supplemental fiber ingredients may include plant based fiber products, either commercially available or developed privately. Examples of some typical fiber products are guar gum, gum arabic, soybean fiber, oat fiber, pea fiber, fig fiber, citrus pulp sacs, hydroxymethylcellulose, cellulose, seaweed, food grade lumber or wood pulp, hemicellulose, etc. Other supplemental fiber ingredients may be derived from grains or grain products. The concentrations of these other fiber raw materials typically range from 0 up to 30 percent, by weight, and more preferably from 10 to 30 percent by weight.

Typical sweeteners may include, but are not limited to, natural sugars derived from corn, sugar beet, sugar cane, potato, tapioca, or other starch-containing sources that can be chemically or enzymatically converted to crystalline chunks, powders, and/or syrups. Also sweeteners can consist of artificial or high intensity sweeteners, some of which are aspartame, sucralose, stevia, saccharin, etc. The concentration of sweeteners may be between from 0 to 50 percent by weight, of the formula, and more preferably between about 1 and 5 percent by weight.

Typical flavors can include, but are not limited to, artificial and/or natural flavor or ingredients that contribute to palatability. The concentration of flavors may range, for example, from 0 up to 15 percent by weight, of the formula. Colors may include food grade artificial or natural coloring agents having a concentration ranging from 0 up to 10 percent by weight, of the formula.

Typical nutritional ingredients may include vitamins, minerals, trace elements, herbs, botanical extracts, bioactive chemicals and compounds at concentrations from 0 up to 10 percent by weight. Examples of vitamins one can add to the fiber composition include, but are not limited to, vitamins A, B1 through B12, C, D, E, Folic Acid, Pantothenic Acid, Biotin, etc. Examples of minerals and trace elements one can add to the fiber composition include, but are not limited to, calcium, chromium, copper, cobalt, boron, magnesium, iron, selenium, manganese, molybdenum, potassium, iodine, zinc, phosphorus, etc. Herbs and botanical extracts include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-camitine, creatine, lycopene, etc.

The juice and pulp can be dried using a variety of methods. The juice and pulp mixture can be pasteurized or enzymatically treated prior to drying. The enzymatic process begins with heating the product to a temperature between 75° F. and 135° F. It is then treated with either a single enzyme or a combination of enzymes. These enzymes include, but are not limited to, amylase, lipase, protease, cellulase, bromelin, etc. The juice and pulp may also be dried with other ingredients, such as those described above in connection with the high fiber product. The typical nutritional profile of the dried juice and pulp is 1 to 20 percent moisture, 0.1 to 15 percent protein, 0.1 to 20 percent fiber, and the vitamin and mineral content.

The filtered juice and the water from washing the wet pulp are preferably mixed together. The filtered juice may be vacuum evaporated to a brix of 40 to 70 and a moisture of 0.1 to 80 percent, more preferably from 25 to 75 percent. The resulting concentrated Morinda citrifolia juice may or may not be pasteurized. For example, the juice would not be pasteurized in circumstances where the sugar content or water activity was sufficiently low enough to prevent microbial growth. It is packaged for storage, transport and/or further processing.

The processed Morinda citrifolia product may also exist as a dietary fiber produced from the fruit puree. Still further, the processed Morinda citrifolia product may also exist in oil form, such as an oil extract. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(cthylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method of treating migraine headaches with a Morinda citrifolia-based formulation without any significant tendency to cause gastric side effects.

### Morinda Citrifolia-Based Naturaceutical Formulations and Methods of Treating Headaches

The present invention features a unique formulation and method of administering the same to treat migraine headaches, or rather advances treatment of migraine headaches by providing a naturaceutical composition or treatment formulated with one or more processed Morinda citrifolia products derived from the Indian Mulberry plant. The Morinda citrifolia product is incorporated into various carriers or naturaceutical compositions suitable for in vivo treatment of a patient. For instance, the naturaceutical formulation may be ingested orally, introduced through an intravenous injection or feeding, or otherwise internalized as is appropriate and directed.

As mentioned, migraine headaches result from one form or another of cerebral vasoconstriction contributing to subsequent vasodilatation. According to the present invention, internalizing the naturaceutical formulation comprising one or more processed Morinda citrifolia products, as well as other ingredients if desired, serves to treat headaches by reducing any existing or future potential cerebral vasoconstriction.

The typical duration of time that migraine lasts ranges from about two hours to two days. A migraine headache is most commonly triggered as a result of cerebral vasoconstriction, which is often followed by vasodilatation. Cerebral vasoconstriction and subsequent vasodilatation may be attributed to any one or a number of factors. One such factor is abnormal electrical charges that cause alterations in other areas of the brain, and particularly in the trigeminovascular system, which lead to biochemical changes in the brain that resultantly induces clumping of the platelets, alterations in the size of blood vessels, and release of pain-producing substances in which some are related to serotonin. Several other factors include stress related factors (e.g., anxiety, anger, worry, excitement, shock, depression, overexertion, changes of routine and changes of climate), food related factors (e.g., chocolate, cheese and other dairy products, red wine, fried food and citrus fruits), and sensory related factors (e.g., bright lights or glare, loud noises and intense or penetrating smells, menstruation and contraceptive drugs).

The naturaceutical composition of the present invention comprises one or more of a processed Morinda citrifolia product present in an amount by weight between about 0.01 and 100 percent by weight, and preferably between 0.01 and 95 percent by weight. Several exemplary embodiments of formulations are provided below. However, these are only intended to be exemplary as one ordinarily skilled in the art will recognize other formulations or compositions comprising the processed Morinda citrifolia product.

The processed Morinda citrifolia product is the active ingredient or contains one or more active ingredients, such as Quercetin and Rutin, and others, for treating and relieving existing headaches, particularly migraine headaches, as well as reducing the onset potential of future headaches. Active ingredients may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The naturaceutical composition comprising Morinda citrifolia may be prepared using any known means in the art. In addition, since the naturaceutical composition will most likely be consumed orally, it may contain one or more agents selected from the group consisting of sweetening agents, flavoring agents, coloring agents, preserving agents, and other medicinal agents as directed.

The present invention further features a method of administering a naturaceutical composition to a mammal for the treatment and relief of a migraine headache and to help prevent or reduce the likelihood or onset potential of future migraine headaches. The method for administering a naturaceutical, or the method for treating a migraine headache, comprises the steps of (a) formulating a naturaceutical composition comprising in part a processed Morinda citrifolia product present in an amount between about 0.01 and 95 percent by weight, wherein the composition also comprises a carrier, such as water or purified water, and other natural or artificial ingredients; (b) introducing the naturaceutical composition into the body, such that the Morinda citrifolia is sufficiently internalized; (c) repeating the above steps as often as necessary to provide an effective amount of the processed Morinda citrifolia to the tissues of the body of the patient to reduce vasoconstriction.

The step of introducing the naturaceutical composition into the body comprises one of ingesting the composition orally. Ingesting the naturaceutical orally means the naturaceutical composition may be formulated as a liquid, gel, solid, or some other type that would allow the composition to be quickly digested and concentrated within the colon. It is important to note that the step of administering the naturaceutical composition should be carried out in an effective manner so that the greatest concentration of naturaceutical composition, and particularly the processed Morinda citrifolia product, is absorbed into the tissues of the patient's body. For the naturaceutical composition to take effect, it must be sufficiently internalized. Once sufficiently internalized, it may then begin to function to treat or relieve an existing migraine headache and its associated conditions or symptoms, and to reduce the onset potential of future migraine headaches as described above.

In one embodiment, the naturaceutical composition is administered by taking between 1 teaspoon and 2 oz., and preferably 2 oz., of the naturaceutical composition every two hours each day, or at least twice a day. Also, the naturaceutical composition is to be taken on an empty stomach, meaning at a period of time at least two hours prior to consumption of any food or drink. Following this, the naturaceutical composition is sufficiently allowed to absorb into the tissues of the body, and particularly within the cerebral area, to actively impact the tissue within the cerebrum to reduce vasoconstriction and convert certain brain receptor proteins and other enzymes into active sites for better and more efficient absorption of various hormones, as well as to assist other proteins in forming pores through membranes in the blood vessels and other organs in the body, each as explained in greater detail below. Of course, one ordinarily skilled in the art will recognize that the amount of composition and frequency of use may vary from individual to individual.

In addition, the step of administering the naturaceutical composition may include injecting the composition into the body using an intravenous pump. This technique is advantageous as it would allow the composition to be localized in the area where it would have the most effect, or the area that would provide for the greatest concentration of the naturaceutical composition.

The treatment of migraine headaches by reducing cerebral vasoconstriction, and commonly associated vasodilatation, results from the affect of these processed Morinda citrifolia products, and/or the active ingredients found therein, namely Quercetin, Rutin, Xeronine, and the building blocks to Xeronine—Proxeronase and Proxeronine. Specifically, the processed Morinda citrifolia products, whether they be in the form of fruit juice, puree juice, dietary fiber, oil, etc., function to convert certain brain receptor proteins and other enzymes into active sites for better and more efficient absorption of various hormones. The processed Morinda citrifolia products also assist other proteins in forming pores through membranes in the blood vessels and other organs in the body. As these products are internalized into the body through the introduction of the naturaceutical formulation in which they are contained, they are absorbed by the various proteins in the body and go to work at facilitating the change in shape of the pores, thus positively or beneficially affecting the passage of molecules through the membranes. These unique affects specifically function to reduce and inhibit the overall effects that the above-mentioned factors have on the body in contributing to or directly causing a migraine headache. Specifically, these affects, or rather the processed Morinda citrifolia products, function to reduce vasoconstriction within the cerebrum, thus providing for a more natural and even flow of blood through the blood vessels existing within the cerebrum. As such, it can be said that the present invention further comprises a method for normalizing or improving the flow of blood within the cerebrum.

The naturaceutical composition described above, and particularly the processed Morinda citrifolia products, also function to reduce and/or relieve many or all of the conditions and symptoms commonly associated with migraine headaches. Depending upon the particular type of migraine headache, namely classical or common, a person can experience different types of conditions symptoms. Classical migraines are associated with objective prodromal neurological signs and symptoms that are preceded by a slowly expanding area of blindness surrounded by a sparkling edge that increases to involve up to one half of the field of vision of each eye. When the area of blindness disperses, after approximately 20 minutes, it is often followed by a severe one-sided migraine headache. Still further secondary symptoms may be experienced, such as nausea, vomiting and an increased sensitivity to light. The common migraine is an attack without prodromal symptoms and begins as a slowly developing pain in the form of a headache that transforms into a mounting throbbing pain made worse by the slightest movement or noise. The pain is often on one side of the head only and usually occurs with nausea and sometimes vomiting. The present invention naturaceutical, and the methods of administering, further comprises treating such conditions or symptoms.

The following tables illustrate or represent some of the preferred formulations or compositions of the naturaceutical as contemplated by the present invention. It should be noted that these formulations are only intended as exemplary embodiments and are not to be construed as limiting in any way.

Formulation One

| Ingredients | Percent by Weight |
|---|---|
| Morinda citrfolia puree juice or fruit juice | 100% |

Formulation Two

| Ingredients | Percent by Weight |
|---|---|
| Morinda citrifolia fruit juice | 85-99.99% |
| water | 0.1-15% |

Formulation Three

| Ingredients | Percent by Weight |
|---|---|
| Morinda citrifolia fruit juice | 85-99.99% |
| non-Morinda citrifolia-based fruit juices | 0.1-15% |

Formulation Four

| Ingredients | Percent by Weight |
|---|---|
| Morinda citrifolia fruit juice | 50-90% |
| water | 0.1-50% |
| non-Morinda citrifolia-based fruit juices | 0.1-30% |

Formulation Five

| Ingredients | Percent by Weight |
|---|---|
| Morinda citrifolia puree juice | 85-99.9% |
| water | 0.1-15% |

Formulation Six

| Ingredients | Percent by Weight |
|---|---|
| Morinda citrifolia puree juice | 85-99.9% |
| non-Morinda citrifolia-based fruit juices | 0.1-15% |

Formulation Seven

| Ingredients | Percent by Weight |
|---|---|
| Morinda citrifolia puree juice | 50-90% |
| water | 0.1-50% |
| non-Morinda citrifolia-based fruit juices | 0.1-30% |

Formulation Eight

| Ingredients | Percent by Weight |
|---|---|
| Morinda citrifolia dietary fiber | 0.1-30% |
| water | 1-99.9% |
| non-Morinda citrifolia-based fruit juices | 1-99.9% |

Formulation Nine

| Ingredients | Percent by Weight |
|---|---|
| Morinda citrifolia dietary fiber | 0.1-30% |
| water | 1-99.9% |
| Morinda citrifolia fruit juice or puree juice | 1-99.9% |

Formulation Ten

| Ingredients | Percent by Weight |
|---|---|
| Morinda citrifolia puree juice concentrate or fruit | 100% |
| juice concentrate | |

Formulation Eleven

| Ingredients | Percent by Weight |
|---|---|
| Morinda citrifolia fruit juice concentrate or puree | 85-99.99% |
| juice concentrate | |
| water | 0.1-15% |

In one preferred method, a person suffering from a migraine headache as described above takes at least one ounce of Formulation One in the morning on an empty stomach, and at least one ounce at night on an empty stomach, just prior to retiring to bed. In one example, which is not meant to be limiting in any way, the beneficial Morinda Citrifolia is processed into Tahitian Noni® juice as manufactured by Morinda, Incorporated of Orem, Utah.

The present invention further features taking a migraine medication concurrently with the naturaceutical formulation. Migraine medications used to treat headache pain are well known and can be grouped into three different categories—symptomatic relief, abortive therapy, and preventive therapy. Symptomatic relief medications are used to relieve symptoms associated with headaches, including the pain of a headache or the nausea and vomiting associated with migraines. Many of these medications are available over-the-counter, without a doctor's prescription. Other medications require a prescription. Abortive medications, when used at the first sign of a migraine, can stop the process that causes the headache pain. By stopping the headache process, abortive medications help prevent the symptoms of migraines including pain, nausea, and sound and light sensitivity. Preventive medications, prescribed to take on a daily basis, are used to treat very frequent tension headaches and migraines, or the combination of both types of headaches. Preventive treatment is aimed at reducing both the frequency and severity of the headaches. To be effective, all preventive medications must be taken one or more times every day.

Taking or administering the naturaceutical formulation comprising one form or another of a processed Morinda citrifolia product as taught and described herein functions to enhance the relief potential for the patient by increasing or enhancing the efficacy of the migraine medication, as well as providing the same benefits and advantages to the patient that are obtained directly from the naturaceutical formulation. The ability of the naturaceutical formulation, and particularly the Morinda citrifolia product contained therein, to increase the efficacy of the migraine medication is explained in further detail in example provided below.

The following examples set forth and present the effects of Morinda citrifolia on both pre-existing headaches, on symptoms and conditions associated with migraine headaches, as well as the preventative effects of Morinda citrifolia against the onset of future headaches. These examples are not intended to be limiting in any way, but are merely illustrative of the beneficial, advantageous, and remedial effects of Morinda citrifolia on headaches, and particularly migraine headaches. Other non-limiting examples of the present invention are described below.

### EXAMPLE ONE

This study was a field study under the direction of a license physician. In the present example, a patient experiencing and diagnosed with an established migraine headache was desirous to treat the migraine headache with a non-prescription, over-the-counter remedy or preparation. Thus, to treat the migraine headache, the individual was given an identified, prescribed amount of a naturaceutical composition to consume orally, wherein the naturaceutical comprised 100% processed Morinda citrifolia fruit juice. The naturaceutical was administered in a safe, pre-determined amount and was administered intermittently a safe, pre-determined number of times, which alleviated the migraine headache and the symptoms associated with the headache, including pain and blocked vision. In addition, the Morinda citrifolia-based naturaceutical was consumed by the patient on an empty stomach.

### EXAMPLE TWO

In this study, several patients (see below for details) who have, on occasion, experienced migraine headaches, were administered orally a serving of one teaspoon of a naturaceutical comprising processed Morinda citrifolia fruit juice, and specifically, Tahitian Noni® fruit juice as manufactured and produced by Morinda, Inc. of Orem Utah. Tahitian Noni comprises Morinda citrifolia fruit juice from fruit puree, grape juice concentrate, blueberry concentrate, and other natural flavorings.

The naturaceutical was administered twice a day for three days on an empty stomach. On the fourth day, the serving size was increased to one tablespoon, and the patients were administered the one ounce serving of the naturaceutical twice a day for three more days, also on an empty stomach. On the 7^{th} day, the serving size was increased to one ounce, and was administered to the patients twice a day for three more days. On the 10^{th} day, the serving size was increased to two ounces, and was administered to the patients twice a day on an empty stomach. The patients were also given a minimum of eight ounces of water each day to consume.

This prescribed dosage comprising the identified serving size and frequency of consumption was maintained thereafter. The patients also were instructed to keep and maintain a diary or log of any pain and symptoms associated with migraine headaches, as well as any other experienced effects resulting from ingestion of the naturaceutical while they were undergoing the study and while they were on the above-prescribed dosage. The pain and associated symptoms were given quantifiable measurements on a scale from 1 to 10, with a 1 indicating that no pain or symptoms were experienced, and a 10 indicating that unbearable pain and symptoms were experienced.

The following table represents a summary of the patient subjects used in the experiment:

| | |
|---|---|
| Total number of patient subjects | 123 |
| Number of patient subjects experiencing a | 40 (34 women, 6 men) (34% |
| pre-existing or an established migraine | of the total) |
| headache | |
| Number of dropouts: | 2 (5% of the total) |
| headache worsened: 1 | |
| allergic reaction: 1 | |

Upon further analysis during and upon conclusion of the experiment of those patients who had been diagnosed with a pre-existing migraine headache, several reported dissipation of their headache and a significant decrease in the amount of pain and symptoms experienced at various rates. Still others reported that their headache never returned after taking the prescribed naturaceutical.

One patient reported that after sixteen weeks of sustained consumption of the prescribed dosage of the naturaceutical that they were headache free.

Moreover, nearly all of the patients who were taking doctor prescribed medications for their migraine headaches, and who supplemented these medications with the Tahitian Noni naturaceutical, reported one or more of the following:

a. decreased pain, decrease sensory symptoms, such as burning, tingling, and numbness, better sleep, and an increase in energy throughout the day;

b. dramatic results in dissipation of existing headaches and reduction in associated symptoms, as well as reducing or eliminating the onset of future migraine headaches, as opposed to the effects experienced from taking their doctor prescribed medications alone;

c. after 3-6 months of maintaining the prescribed dosage, many migraine patients reported that they no longer experienced migraine headaches;

d. many patients tapered off use of their other doctor prescribed medications;

e. some patients were able to return to work and school while others took some vacations; and

f. many migraine patients reported good results in 1-2 weeks and some in a matter of just three days.

All patients were regularly interviewed for follow up. In addition, all patients who were involved in the study were clinically diagnosed as having migraine headache by a licensed medical physician, who also took part in the follow up interviews.

Overall, those patients who followed the prescribed dosage of taking the above-described Morinda citrifolia-based naturaceutical reported better sleep, increased energy, less pain with some reporting no pain, stabilized mood with less depression, diminished need for narcotic medication, and decreased sensory symptoms.

Of important note, no side effects were reported except for one patient that reported their migraine worsened, which may be attributed to external environmental conditions. One other patient had a negative reaction (hives) and withdrew from the study.

### EXAMPLE THREE

It is well established that the chemical serotonin is involved in the migraine headache pathways. Serotonin is a neurotransmitter that assists in the communication between the brain and the body. Those who suffer from migraine headaches have lower levels of serotonin as revealed by numerous studies and clinical trials alike. Thus, many medications have been formulated that function to reduce or prevent the lowering of serotonin levels in the body, which subsequently helps relieve the pain associated with migraine headaches. These medications contain one form or another of serotonin antagonist receptors. For example, serotonin antagonist receptors 5HT-1 and 5HT-2 are implicated in the pathways in which levels of serotonin can be enhanced.

As migraine symptoms worsen, 5-HT levels decrease. Intracranial blood vessels dilate resulting in a decrease in blood flow. Left alone, symptoms naturally improve and the migraine attack stops as a result of the return of generated 5-HT. On the other hand, stimulation of 5-HT type 1 receptors by migraine medications functions to hasten recovery or relief of a migraine attack. 5-HT agonists seem ineffective during migraine aura and fail to correct brainstem pathology as seen in positron emission tomography (PET) scans. As such, migraine headache recurrence may be as high as 50 percent using 5-HT agonists alone.

According to the present invention, processed Morinda citrifolia products as described and taught herein, and particularly processed Morinda citrifolia puree and puree juice concentrates, comprise the following serotonin inhibition:

| | | |
|---|---|---|
| 5-HT2A | 10% | 103% Inhibition |
| | 5% | 103% Inhibition |
| | 2.5% | 93% Inhibition |
| IC₅₀ <2.5% | | |

This offers some explanation of why taking one form or another of a processed Morinda citrifolia product as taught herein increases the efficacy of doctor prescribed migraine medications taken by sufferers of migraine headaches as noted above.

Moreover, processed Morinda citrifolia puree and puree juice is very active against cytochrome enzymes CYP450, which are responsible for the breaking down or metabolizing of drugs. The following enzymes were analyzed and their percentage of effectiveness reported:

| | |
|---|---|
| CYP450, 1A2 | 81% |
| CYP450, 2C19 | 89% |
| CYP450, 2C9 | 81% |
| CYP450, 2D6 | 93% |
| CYP450, 3A4 | 77% |

These results indicate that the present invention processed Morinda citrifolia products prevents or significantly reduces the breaking down or metabolizing of migraine medications or drugs by inhibiting the drug breakdown functions of the cytochrome enzymes. As a result, drugs are allowed to be in the body much longer, which naturally allows them to be more effective.

The present invention may be embodied in other specific forms without departing from its spirit of essential characteristics. The described embodiments are to be considered in all respects only al illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A naturaceutical formulation for treating headaches and its associated symptoms in mammals, said naturaceutical formulation comprising a processed Morinda citrifolia product;

2. The formulation of claim 1, wherein said processed Morinda citrifolia product comprises Morinda citrifolia fruit juice.

3. The formulation of claim 1, wherein said processed Morinda citrifolia product comprises Morinda citrifolia puree juice.

4. The formulation of claim 1, wherein said processed Morinda citrifolia product comprises Morinda citrifolia puree juice concentrate.

5. The formulation of claim 1, wherein said processed Morinda citrifolia product comprises Morinda citrifolia fruit juice concentrate.

6. The formulation of claim 1, wherein said processed Morinda citrifolia product comprises Morinda citrifolia dietary fiber.

7. The formulation of claim 1, wherein said processed Morinda citrifolia product of said naturaceutical formulation further comprises an active ingredient Quercetin present in an amount between about 0.1 and 10 percent by weight.

8. The formulation of claim 1, wherein said processed Morinda citrifolia product is present in an amount between about 0.01 and 100 percent by weight.

9. The formulation of claim 7, wherein said processed Morinda citrifolia product further comprises Rutin as an additional active ingredient that synergistically works with said Quercetin to treat said migraine headaches and its associated symptoms.

10. The formulation of claim 9, wherein said Rutin is present in an amount between about 0.1 and 10 percent by weight.

11. The formulation of claim 1, wherein said naturaceutical is administered orally.

12. The formulation of claim 1, wherein said naturaceutical is administered transdermally to said infected area.

13. The formulation of claim 1, wherein said naturaceutical is administered intravenously.

14. The formulation of claim 1, wherein said naturaceutical is administered systemically.

15. A method of treating headaches and its associated symptoms in mammals, said method comprising the steps of:
obtaining a naturaceutical formulation comprising a processed Morinda citrifolia product; and
orally administering a safe, pre-determined amount of said naturaceutical formulation for safe, pre-determined frequency for a safe, pre-determined duration.

16. The method of claim 15, wherein said processed Morinda citrifolia product comprises Morinda citrifolia fruit juice.

17. The method of claim 15, wherein said processed Morinda citrifolia product comprises Morinda citrifolia puree.

18. The method of claim 15, wherein said processed Morinda citrifolia product comprises Morinda citrifolia puree juice.

19. The method of claim 15, further comprising the step of supplementing said step of orally administering said food product with a prescribed migraine medication formulated for the treatment of migraine headaches, said migraine medicine administered concurrently with said naturaceutical.

20. The method of claim 15, wherein said safe, pre-determined amount is between about one teaspoon and about two ounces.

21. The method of claim 15, wherein said safe, pre-determined frequency comprises at least two times per day on an empty stomach.

22. The method of claim 15, wherein said safe, pre-determined duration of time comprises at least three consecutive days.

23. A method for preventing the onset of a future headache in a mammal, said formulation comprising:
obtaining a naturaceutical formulation comprising a processed Morinda citrifolia product; and
orally administering to said mammal between about 1 teaspoon and 2 ounces of said naturaceutical formulation at least twice daily on an empty stomach each day.

24. A method for treating a migraine headache, said method comprising the steps of:
orally administering at least one ounce of a naturaceutical formulation comprising processed Morinda citrifolia fruit juice on an empty stomach in the morning;
orally administering at least one ounce of said naturaceutical formulation prior to sleeping at night; and
repeating said steps of orally administering for continued consecutive days.

25. The method of claim 24, wherein said naturaceutical formulation comprises:
processed Morinda citrifolia fruit juice present in an amount by weight of about 100 percent.

26. The method of claim 24, wherein said naturaceutical formulation comprises:
processed Morinda citrifolia fruit juice present in an amount by weight between about 85-99.99 percent; and
water present in an amount by weight between about 0.1-15 percent.

27. The method of claim 24, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

28. The method of claim 24, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0.1-30 percent.

29. A method for reducing cerebral vasoconstriction and subsequent vasodilatation contributing to a migraine headache, said method comprising the steps of:
administering to a patient a naturaceutical formulation comprising a processed Morinda citrifolia product.

30. The method of claim 29, wherein said step of administering comprises orally ingesting at least one ounce of said naturaceutical formulation twice daily on an empty stomach.

31. The method of claim 29, wherein said step of administering comprises intravenous injection of said naturaceutical formulation.

32. The method of claim 1, wherein said step of administering comprises transdermal delivery of said naturaceutical formulation.

33. The method of claim 29, wherein said processed Morinda citrifolia product comprises Morinda citrifolia fruit juice.

34. The formulation of claim 29, wherein said processed Morinda citrifolia product comprises Morinda citrifolia puree juice.

35. The formulation of claim 29, wherein said processed Morinda citrifolia product comprises Morinda citrifolia puree juice concentrate.

36. The method of claim 29, wherein said processed Morinda citrifolia product comprises Morinda citrifolia fruit juice concentrate.

37. The method of claim 29, wherein said processed Morinda citrifolia product comprises Morinda citrifolia dietary fiber.

38. A method for normalizing or improving the flow of blood within the cerebrum during a migraine attack, said method comprising the steps of:
reducing vasoconstriction in said cerebrum through the prophylactic administration of a naturaceutical composition comprising a processed Morinda citrifolia product present in an amount by weight between 0.1 and 100 percent.

39. The method of claim 38, further comprising the step of reducing vasodilatation through the prophylactic administration of said naturaceutical composition.

40. A method for treating prodromal neurological signs and symptoms associated with a headache comprising the steps of:
orally administering at least one ounce of a naturaceutical formulation comprising a processed Morinda citrifolia product on an empty stomach in the morning;
orally administering at least one ounce of said naturaceutical formulation prior to sleeping at night; and
repeating said steps of orally administering for continued consecutive days.

41. A method of maintaining and enhancing serotonin levels in the body of a mammal for the purpose of treating migraine headaches, said method comprising the steps of:
orally administering at least one ounce of a naturaceutical formulation comprising a processed Morinda citrifolia product on an empty stomach in the morning;
orally administering at least one ounce of said naturaceutical formulation prior to sleeping at night; and
repeating said steps of orally administering for continued consecutive days.

42. A method for inhibiting the drug breakdown functions of cytochrome enzymes for the purpose of treating a migraine headache, said method comprising the steps of:
administering a migraine medication to a patient;
administering a naturaceutical to said patient concurrently with said migraine medication, said naturaceutical comprising a processed Morinda citrifolia product that reduces the breaking down or metabolizing of said migraine medication by inhibiting the medication breakdown functions of said cytochrome enzymes, thus increasing the efficacy of said migraine medication, said processed Morinda citrifolia product present in an amount by weight between 20 and 100 percent.

43. The method of claim 42, wherein said migraine medication is selected from the type consisting of symptomatic, abortive, and preventative.

44. The method of claim 42, wherein said processed Morinda citrifolia product comprises processed Morinda citrifolia fruit juice.

45. The method of claim 42, wherein said processed Morinda citrifolia product comprises processed Morinda citrifolia puree juice.

46. The method of claim 42, wherein said processed Morinda citrifolia product comprises processed Morinda citrifolia dietary fiber.

47. The method of claim 42, wherein said processed Morinda citrifolia product comprises processed Morinda citrifolia fruit juice concentrate.

48. The method of claim 42, wherein said processed Morinda citrifolia product comprises processed Morinda citrifolia puree juice concentrate.
